(19) United States
(12) Patent Application Publication
(10) Pub. No.: US 2021/0393839 A1
(43) Pub. Date: Dec. 23, 2021

(54) Tapered indicator to be used in process challenge devices

(71) Applicant: GKE GmbH, Walderns (DE)

(72) Inventor: Ulrich KAISER, Glashütten (DE)

(73) Assignee: GKE GmbH, Waldems (DE)

(21) Appl. No.: 17/292,785

(22) PCT Filed: Nov. 12, 2018
(86) PCT No.: PCT/EP2018/080902

§ 371 Date: May 11, 2021

(51) Int. Cl. A61L 2/28; G01N 31/22

(52) U.S. Cl. CPC A61L 2/28; G01N 31/226

(57) ABSTRACT

Indicator (22) for process challenge devices (PCDs) to monitor sterilizing processes, comprising a plurality of indicator areas (50, 54, 58, 62, 66) which react with a sterilizing agent and which are arranged spaced apart on a common carrier element (18) in a longitudinal direction (70) of said carrier element (18), whereby said carrier element (18) is built tapered along its longitudinal direction (70).

### FIELD OF THE INVENTION

The invention refers to an indicator for process challenge devices (PCDs), especially simulating the worst-case penetration conditions of a load inside a sterilization chamber. It also refers to the design of an indicator sheet to easily manufacture the tapered indicator and to be used in different process challenge device.

### BACKGROUND

For aseptic operations in hospitals and sterile filling in industry the use of sterile operating instruments or materials is absolutely necessary. At a therefore necessary sterilization the sterilizing agent, for example steam, formaldehyde, ethylene oxide, hydrogen peroxide and/or ozone, is usually transferred via the gas phase to the surface of the instrument to be sterilized, to assure the total inactivation of existing germs. For this purpose, sterilizers with sterilization chambers are normally used in which the instruments or materials—always packaged—which have to be sterilized, are put in. For the real sterilization the sterilization chamber requires first air removal and then is flooded with gaseous sterilant—also named sterilizing agent—, which requires that air inside has to be removed before. The sterilization agent shall contact the surfaces of the instruments or materials to be sterilized so that the desired kill of the germs occurs.

As the complete sterilization of the goods at all surface areas is only guaranteed if the sterilization agent reaches all interior surfaces as well, for example porous packs or hollow devices, like tubes and minimal invasive instruments. The removal of air inside the goods and inside the sterilization chamber has to be secured by a suitable air removal procedure before the sterilization process starts. After that the sterilization chamber is flooded with the sterilization agent in order to reach all surfaces of the instruments inside the sterilization chamber with the sterilization agent. This is only possible when the complete penetration of the sterilization agent is assured to all surfaces through the packaging and geometric design of the instruments.

Sterilization agent is preferably steam, formaldehyde, ethylene oxide or H₂O₂.

The complex structure of minimal invasive surgical (MIS) instruments is known to be problematic in sterilization processes. More and more medical devices are used with relatively long pipes or tubes and comparatively small free cross sections, so that a reliable surface contact of the sterilization agent to all interior surfaces becomes more and more difficult if there are remaining inert gases, like air, present. Furthermore, materials and goods with complex interior surfaces, for example textile packages, require sterilization as well. In such cases existing accumulations of remaining air or other non-condensable gases (NCG) may prevent complete or part contact of those surfaces.

The complete sterilization is only assured when the air inside the goods is completely removed before the sterilization process, and/or, when during the vacuum stage no air enters through leaks and/or no NCG are introduced into the sterilization chamber with the sterilization agent, to assure that the sterilization agent can reach all surfaces.

As instruments are packed and therefore sterility cannot be tested directly before use, the validation of sterilization processes before start up and routine monitoring during the sterilization process are necessary. In addition, detectors are used to prove the success of the sterilization process. For example chemical indicators are used which change their color when the indicator has been exposed to all critical variables and their critical parameters of the sterilization process, for example with condensing steam and temperature over time. Alternatively or additionally, biological indicators may be used in form of stripes, suspensions or self-contained biological indicators (SCBI). After a sterilization process sterility has to be tested to release the load.

Such chemical or biological indicators monitor all critical sterilization variables and their parameters at the place where the indicator inside the sterilization chamber is positioned. Such indicators cannot be placed at those critical areas of inaccessible surfaces of complex instruments and therefore there is no direct proof of the sterilization success possible. To circumvent this problem, surrogate test devices are used and are sterilized together with the goods, to determine the success of the sterilization at for the indicator non-accessible places. For example, for the sterilization process of textiles or other materials, from Bowie and Dick a standard test pack was described (Bowie, I. W., e.a., The Bowie+Dick autoclave tape test, LancetI, 1963, p. 585-587), in which a chemical indicator test sheet of DIN A4-size has been placed centrally in a cotton package of 7 kg weight according to EN 285. Though this standard test is not exactly reproducible because of the cotton quality, cotton history and individuality of the packages, and its penetration characteristic is different from hollow devices.

Alternatively, so called process challenge devices (PCDs) or indicator test systems are used. In such a test system, as described for example in EP 0 628 814 A1 or in EN 867-5, the difficult accessible inside surface of complex instruments is simulated by a suitable model, enabling to monitor the success of the penetration processes into complex instruments in an analogous way.

Those well-known indicator systems consisting of a PCD and of a suitable detector to prove the penetration of the sterilization agent, connected to a suitably chosen length of tube at the gas entering side, which is open at its admission end as described in EN ISO standard 11140-1. This indicator system simulates the penetration characteristics of similarly designed instruments which are supposed to be sterilized, where especially during an alternate gas exchange according a fractionated vacuum and/or the condensation of steam eventually remaining air or other non-condensable gases at the tube end in the area of the detector are concentrated.

If the detector of such a system connected to the tube end detects sterilization agent, it can be assumed, that—adding a security supplement according to the Penetration characteristics—the instruments most inaccessible points of their inner surfaces must have been in contact with sterilization agent as well. Such a tube model as a test device which can hold for example biological or chemical indicators as a detector is also intended for the verification of sterilization processes in Euro Standard EN 867-5. To check the sterilization success of more complex instruments, test devices of a different construction which are in their dimension suitably adapted, can be used, as described for example in the Euro Standards EN 285, EN 14180, EN 1422 or EN 867-5 as type tests.

The detector volume is positioned at the closed end of the test device while the entrance to the gas collection volume is arranged, for instance, at the other end of the test device. The detector volume can be opened such that the detector can be placed inside the detector volume and be closed again. When the test device is placed in the sterilisation chamber the detector entrance is exposed to sterilisation agent. In order to prevent direct entry of sterilisation agent into the detector volume, which would nullify the test, a sealing element has to be provided to seal the region where the detector chamber can be opened. The sealing element, which is for example built as a gasket, wears off over time and needs to be replaced. An undetected malfunction leads to non-valid test results. The devices need to be long enough to provide a valid simulation of instruments.

An indicator system is defined according EN ISO 11140-1 as a combination of a process challenge device and a detector inside which can be a biological, chemical or physical indicator as a detector to monitor the presence of the sterilant. It is used as a surrogate model to represent the worst-case penetration conditions inside of a sterilization load including its packaging. Since biological or chemical indicators cannot be put into the worst-case penetration locations inside of complex instruments, it is used as a surrogate model to check if the sterilization conditions are achieved in the load, represented by the surrogate indicator system.

A disadvantage of known indicators is that, after their removal from the process challenge device, it cannot be recognized which end of the indicator was at the dead end or farthest end of the gas pathway, this end corresponding to the most difficult location to be reached by the sterilizing agent.

### SUMMARY OF THE INVENTION

It is an object of the invention is to allow a reliable identification of the indicator portion which is located at the end of the passage of the sterilizing agent. It is a further invention to enhance the accuracy to monitor the success of a sterilizing process.

The first aspect of the invention relates to an indicator for PCDs to monitor sterilizing processes, comprising a plurality of indicator areas which react with a sterilizing agent and which are arranged spaced apart on a common carrier element in a longitudinal direction of the carrier element, whereby the carrier element is built tapered along its longitudinal direction.

Preferred embodiments of the invention are described in the dependent claims and the figure.

The invention is based on the consideration that assessment of the success of a sterilizing process should be assessed with the highest accuracy. It can be considered to be successful if the indicator inside the location in a PCD which is most difficult to reach by the sterilizing agent and has reacted with the sterilizing agent. It would therefore be highly desirable to have the possibility to recognize which end of the indicator is arranged at the very end of the gas passage.

The applicant has recognized that a reliable recognition can be realized by providing an indicator with a tapered form which allows positioning it in the detector chamber of a PCD in a unique orientation. In this way, even after extraction of the indicator from the PCD, it can be reliably determined if the sterilizing agent has reached the most difficult location in the process challenge device.

The information which end of the indicator was located at the end of the gas passage is key or vital information on the sensitivity reached during the sterilization process as it reflects the location which is hardest to reach by the sterilizing agent.

In a preferred embodiment, the carrier element is built flat, whereby the tapered shape realizes a reduction of the width of the carrier element along the longitudinal direction. The carrier element of the indicator can be made by any material and thickness.

The carrier element advantageously tapers in a continuous way.

In a preferred embodiment, the indicator comprises between 2 and 7, especially 5, indicator areas.

Preferably the indicator has a length between 40 and 70 mm, especially 50 mm, and/or has a maximal width between 3 and 8 mm. It has especially a 4 mm small and a 6 mm large width, the width of 6 mm at the large width and 4 mm of small width of the tapered indicator.

The indicator is preferably built as a chemical and/or biological and/or physical indicator. Alternatively, it can be also be built as a combination of 2 or 3 of these indicators. This combination of indicators could be tapered or rectangular.

Advantageously, the carrier element is made of any material, preferably of paper, plastic foil or any metal in flat shape of any thickness, preferably 0.5-1 mm thick or of any other shape.

Preferably, the chemical or physical indicator can be detected by daylight and/or UV-light and/or infrared and/or X-rays and/or y rays with a corresponding detection unit.

In a second aspect, the invention relates to an indicator sheet, on which a plurality of indicator strips is arranged, whereby cuts are arranged on the sheet such that a division of the sheet along these cuts results in a plurality of indicators according to one of the previous claims. The carrier strips with an indicator on which reacts with the sterilizing agent in a detectable way, especially in a visually recognizable way, but it may be also detected with UV-light and/or infrared and/or X- and/or y-rays.

Preferably the cuts comprise lines printed on the sheet. The cuts can be perforated and be separated from the sheet. In a preferred embodiment, between 20 and 30, especially 25, indicators can be taken off from the sheet.

In a third aspect, the invention relates to a PCD comprising a detector chamber with an indicator volume which is configured to receive an indicator previously described, whereby the indicator volume tapers in a longitudinal direction as to fit the indicator. Since the indicator is built in an asymmetric way, the indicator fits into the indicator volume only in one orientation. In this way, a unique identification which indicator end was at the end of the gas passage way becomes possible.

In a preferred embodiment, the detector chamber comprises a dead end, whereby the indicator volume tapers and its narrow or large end is located at this dead end, depending how the PCD is constructed.

In a preferred embodiment, the PCD comprises (at least) two parts, namely a housing part and an insertion part, whereby the insertion part is at least partially insertable into the housing part in such a way that the detector chamber is arranged without any seal inside these both parts.

The PCD can be designed as a tube with a capsule at one end contains a tapered indicator holder/indicator described above.

The PCD can have a different tube length and diameter are connected in series containing a tapered indicator holder/indicator described above.

The advantages of the invention are especially as follows. The asymmetric design of the indicator allows an enhanced assessment of the quality and success of the sterilizing process since it can clearly be determined which end of the indicator was at the far end of the gas passage. By providing a sheet from which indicators can easily be taken off, a convenient packaging of indicators is realized. A PCD with a detector chamber built to receive in a unique orientation such an indicator allows an enhanced assessment of sterilizing processes.

### BRIEF DESCRIPTION OF THE DRAWINGS

Further features and advantages of the present invention shall become clearer from the following detailed description of some of its preferred embodiments, made with reference to the attached schematic drawings and given as an indication and not for limiting purposes.

In particular, the attached drawings are included to provide a further understanding of the invention and are incorporated in and constitute a part of this specification. The drawings together with the description explain the principles of the invention. In the drawings, corresponding characteristics and/or components are identified by the same reference numbers. In these drawings:

FIG. 1 shows an indicator sheet in a preferred embodiment with a plurality of strips;

FIG. 2 shows the sheet according to FIG. 1 in a different representation;

FIG. 3 shows an indicator in a preferred embodiment;

FIG. 4 shows a detector chamber of a process challenge device with an inserted indicator according to FIG. 3; and

FIG. 5 shows a process challenge device in a preferred embodiment.

### DETAILED DESCRIPTION

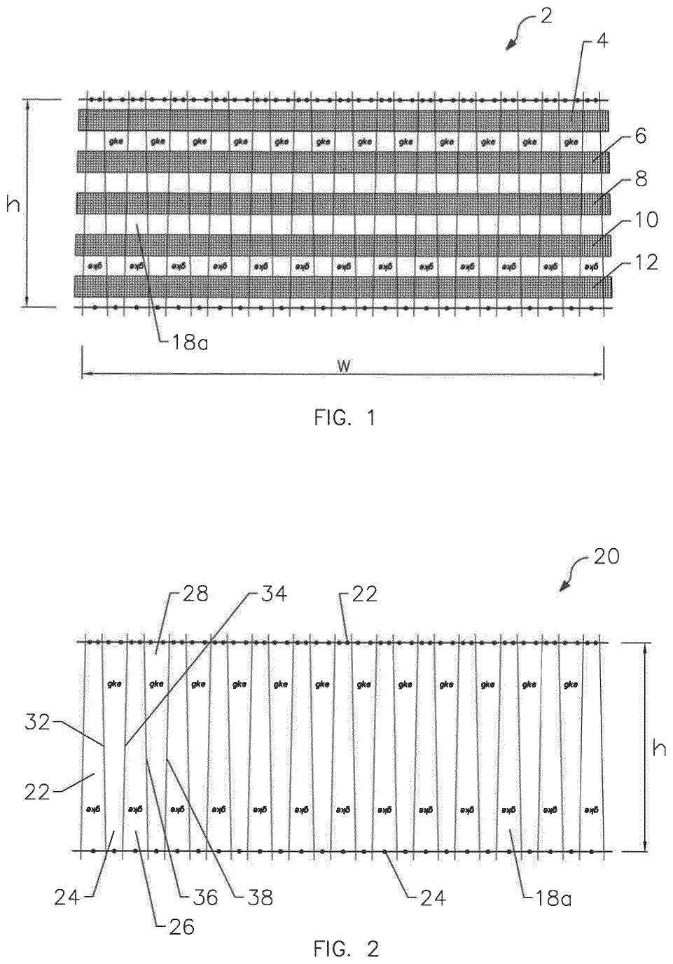

In FIG. 1, an indicator sheet 2 is shown which comprises a support element 18a on which several strips 4, 6, 8, 10, 12 of sensitive indicator colors are arranged. The sheet 2 and therefore also the support element 18a has a rectangular shape. Preferably, the strips 4-12 have, respectively, a rectangular shape and extend over the whole width w of the support element 18a. The strips 4-12 are arranged distanced in the direction of height h of the support element 18a. In the preferred embodiment shown, five strips 4-12 are arranged on the support element 18a.

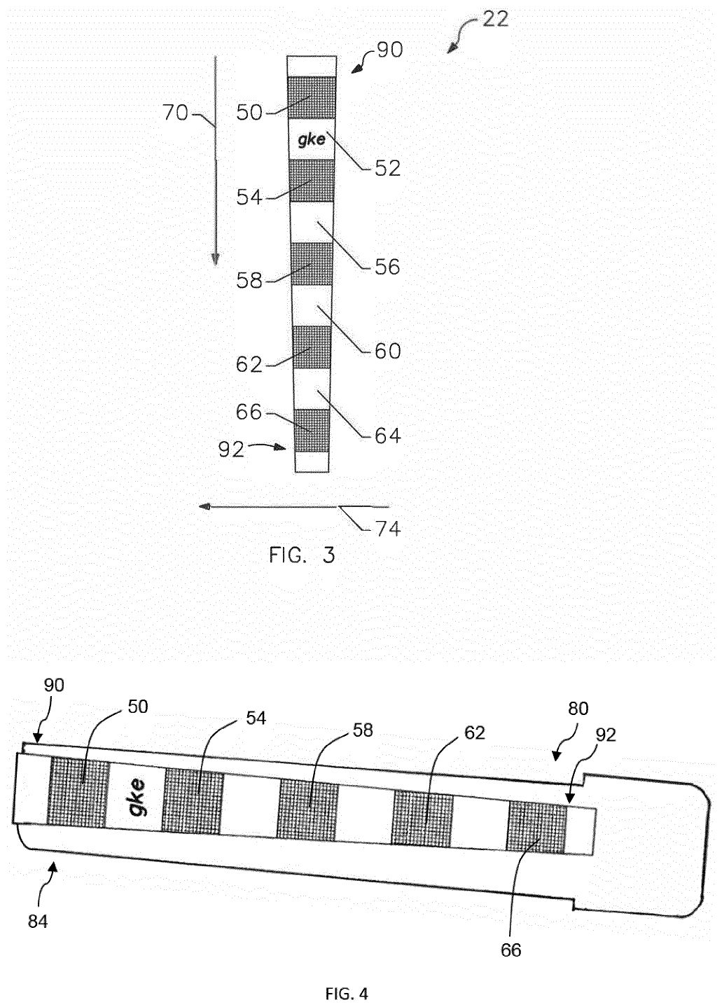

In FIG. 2, the sheet 2 is shown in a different view/representation. On this sheet 2, cuts are arranged which currently comprise lines of which, as an example, lines 32, 34, 36, 38 are labelled. The cuts alternatively are perforations along the respective lines 32-38. The perforations extend along the whole height h of sheet 2 between two borders 22, 24 of sheet 2. When the sheet 2 is divided along perforated line 32 by separating adjacent parts, one indicator 22 (see FIG. 3) is taken off from the sheet 2 and can immediately be used. When sheet 2 is further divided along perforated line 34, a further indicator 24 is taken off. In this way, in the preferred embodiment shown, 25 indicators 22 can be taken off from one sheet 22. The advantage of providing an indicator sheet 2 in this way allows a convenient cutting/perforation/packaging of indicators.

Two adjacent perforated lines, for example lines 34 and 34, are, respectively, inclined towards each other, i.e. they do not run parallel. When the sheet 2 is divided along these adjacent lines, an indicator 22 is taken off which is in longitudinal direction alternating wide and narrow.

An indicator 22 which is taken off from sheet 2 in a preferred embodiment is shown in FIG. 3. The indicator 22 comprises a carrier element 18 which is a part of the support element 18a of indicator sheet 2 which has been separated from the remaining sheet 2. On carrier element 18, several sensitive or indicator areas 50, 54, 58, 62, 66 are arranged which react in response to the presence of a sterilizing agent. These indicator areas 50-66 are, respectively, parts of the strips 4-12 of sheet 2. Between sensitive areas 50-66, regions 56, 60, 64 of the support element 18a are arranged by which the indicator areas 50-66 are spaced apart from each other.

As can be seen in FIG. 3, the width of the indicator 22, i.e. its extension in a lateral direction 74, decreases along longitudinal direction 70. In the preferred embodiment shown, the width decreases continuously, especially conically in two dimensions. In this way, at a first end 90 the indicator 22 has a larger width than at second end 92. The indicator 22 is therefore asymmetrically built and the first end 90 is easily distinguishable from the second end 92.

The indicator 22 is preferably a chemical indicator, whereby the carrier element 18 of the indicator 22 is made from paper, metal, glass fiber, plastic, stainless steel, any plastic foil, Tyvek or any combination. Alternatively, it is a biological indicator strip, whereby the carrier element 18 of the spores is made of paper, metals, glass, glass fiber, plastic or any combination of these materials. The indicator 22 can be surface-protected or covered on both sides with different chemical indicator colors to be used to monitor different sterilization processes.

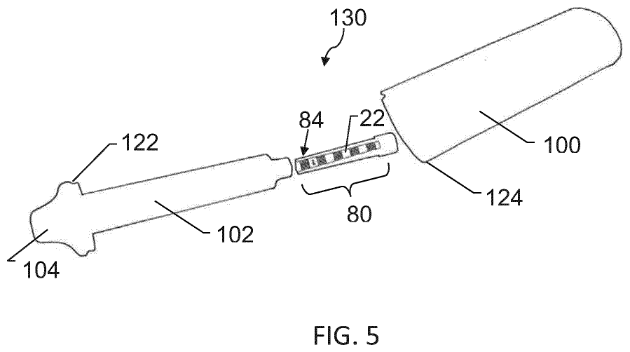

In FIG. 4, a detector chamber 80 of a PCD 130 shown in FIG. 5 is shown which comprises an indicator volume 86. The detector chamber or holder 80 comprises a dead end 84 which is a dead end for a gas passage. The indicator volume 86 is dimensioned to fit the indicator 22 only in one orientation, i.e. the narrow end 92 of indicator 22 is located at the dead or closed end 84.

FIG. 5 shows a process challenge device (PCD) 130 in a preferred embodiment. The PCD 130 in a preferred embodiment comprises a first part 100 or housing part and a second part 102 or insertion part, whereby the second part 102 is insertable into the first part 100. The detector holder 80 shown in FIG. 4 is insertable into second part 102. Then second part 102 which comprises a head part 104 is inserted into first part 100 until an edge 122 of second part 102 is in contact with an edge 124 of first part 100.

The housing and/or said insertion parts are preferably made of metal, plastic or metal-plastic-bonded system.

The head part 104 allows a convenient handling of parts 100, 102, especially insertion and removal of second part 102 into first part 100. After the sterilizing process has finished, the second part 102 is pulled out of first part 100 and the detector chamber 80 is extracted. Since the indicator has an asymmetric shape, especially a conical design, also after extraction of the indicator from the detector chamber 80, it can be reliably determined if the sterilizing agent has reached the dead end of the detector chamber 80 which corresponds to the location of the indicator which is most difficult to reach by the sterilizing agent. In this way, a very accurate assessment of the quality and accuracy of the sterilizing process can be made.

Preferably the two parts 100, 192 fit into each other without sealing and form, combined together, in series connected volumes for providing specific penetration characteristics for sterilization agents. Advantageously, the series of volumes provides connected channels, whereby one end of these channels is connectable to a sterilization chamber and the other end is connected to the detector chamber which is configured to house, alternatively one or more chemical, biological, or physical indicators 22 for detecting the presence of a sterilizing agent.

1. Indicator (22) for process challenge devices (PCDs) to monitor sterilizing processes, comprising a plurality of indicator areas (50, 54, 58, 62, 66) which react with a sterilizing agent and which are arranged spaced apart on a common carrier element (18) in a longitudinal direction (70) of said carrier element (18), characterized in that said carrier element (18) is built tapered along its longitudinal direction (70).

2. Indicator (22) according to claim 1, whereby said carrier element (18) is built flat and whereby said tapering realizes a reduction of the width of said carrier element (18) along said longitudinal direction (70).

3. Indicator (22) according to claim 1 or 2, whereby said carrier element (18) tapers in a continuous way.

4. Indicator (22) according to one of the previous claims, comprising between 2 and 7, especially 5, indicator areas (50, 54, 58, 62, 66).

5. Indicator (22) according to on the previous claims, with a length between 40 mm and 70 mm, especially 50 mm and/or with a maximal width between 4 mm and 8 mm.

6. Indicator (22) according to one of the previous claims, which is built as a chemical and/or biological and/or physical indicator.

7. Indicator (22) according to one of the previous claims, whereby said carrier element (18) is made of any material, preferably of paper, plastic foil or any metal in flat shape of any thickness, preferably 0.5-1 mm thick or of any other shape.

8. Indicator (22) to one of the previous claims, whereby the chemical or physical indicator (22) can be detected by daylight and/or UV-light and/or infrared and/or X-rays and/or y rays with a corresponding detection unit.

9. Indicator sheet (2), on which a plurality of strips (4-14) is arranged, characterized in cut lines arranged on said sheet (2) such that a division of said sheet (2) along said cuts results in a plurality of indicators (22) according to one of the previous claims.

10. Sheet (2) according to claim 9, whereby said cut lines are replaced by perforated lines along which said sheet (2) can be separated and taken out.

11. Sheet (2) according to claim 9 or 10, whereby between 15 and 30, especially 25, indicators can be separately taken out from said sheet (2).

12. Process challenge device (130), comprising a detector chamber (80) with an indicator volume (86) configured to receive an indicator (22) according to one of the previous claims, characterized in that said indicator volume (86) tapers in a longitudinal direction as to fit said indicator (22).

13. Process challenge device (130) according to claim 12, whereby said detector chamber (80) comprises a dead end (84), and whereby said indicator volume (86) tapers in such a way that its wide end is located at said dead end (84), especially which depends on the PCD constructions.

14. Process challenge device (130) according to claim 12 or 13, comprising two parts (100, 102), namely a housing part (100) and an insertion part (102), whereby said insertion part (102) is at least partially insertable into said housing part (100) in such a way that said detector chamber (80) is arranged without any seal inside said both parts (100, 102).

15. Process Challenge Device (130) according to one of the claims 12 to 14, designed as a tube with a capsule at one end contains a tapered indicator holder according to claims 1 to 8.
